(19) United States
(12) Patent Application Publication
(10) Pub. No.: US 2015/0289992 A1
(43) Pub. Date: Oct. 15, 2015

(54) APPARATUS AND METHOD FOR POSITIONING OF ACETABULAR COMPONENTS DURING HIP ARTHROPLASTY PROCEDURES

(71) Applicant: UTI Limited Partnership, Calgary (CA)

(72) Inventors: Carolyn Ruth Anglin, Calgary (CA); Mohsen Akbari Shandiz, Calgary (CA); James MacKenzie, Calgary (CA); Barry Dean Wylant, Calgary (CA); John Gunnar Person, Calgary (CA); Karen Cherk Ting Ho, Calgary (CA)

(73) Assignee: UTI Limited Partnership, Calgary (CA)

(21) Appl. No.: 14/437,693

(22) PCT Filed: Oct. 21, 2013
(86) PCT No.: PCT/CA2013/000895

§ 371 Date: Apr. 22, 2015

Related U.S. Application Data
(60) Provisional application No. 61/717,025, filed on Oct. 22, 2012.
(60) Provisional application No. 61/771,599, filed on Mar. 1, 2013.

(51) Int. Cl. A61F 2/46

(52) U.S. Cl. CPC A61F 2/4609; A61F 2/4657; A61F 2/4684; A61F 2002/4627; A61F 2002/4628; A61F 2002/4662; A61F 2002/4687

(57) ABSTRACT

The present disclosure pertains to apparatus and methods for positioning the angular orientation and depth positioning of an acetabular component during hip arthroplasty procedures. The apparatus comprises a positioning member and guiding member set according to a preoperatively determined angular orientation derived from a pelvic radiograph or radiographs in accordance with the methods provided.

### FIELD OF THE INVENTION

The present disclosure pertains to the field of surgical devices and more specifically, to a method and apparatus for acetabular component positioning during hip arthroplasty.

### BACKGROUND OF THE INVENTION

Malpositioning of the acetabular component during hip arthroplasty can lead to a number of problems including hip instability, dislocation, wear, loosening, impingement, and reduced range of motion. As a result, acetabular component malpositioning is the single greatest factor determining the likelihood of both early and late revision hip arthroplasty. Accurate positioning of the acetabular component is, therefore, crucial to the success of hip arthroplasty.

Angular orientation of the acetabular component with respect to both inclination and anteversion has been identified as the key factor in accurate positioning. The optimal ranges of inclination and anteversion of the acetabular component are considered to be between 30 to 50 degrees of inclination, and 5 and 25 degrees of anteversion. Techniques for orienting inclination and anteversion during hip arthroplasty typically rely on positioning the acetabular component to reference landmarks.

Both anteversion and inclination are judged from the “anterior pelvic plane” (APP), which is made up of the two anterior-superior-iliac-spines (ASIS) and the two pubic tubercles (PT). These are difficult to palpate reliably when there is a large fat layer. Usually in surgery, the patient, lying on their side, abuts a holder, and the surgeon assumes the APP is perpendicular to the table, but this can vary greatly due to the extra layers of tissue, and the pelvis can move considerably as the surgeon is working (forcefully) on the hip. The range of resulting anteversion and inclination is therefore great. Typically over 50% of cups are placed outside of the recognized safe zone. Accordingly, accurate referencing to the pelvis during the insertion of the socket is challenging.

Various techniques have been devised to assist the surgeon in overcoming the challenges associated with determining the angular orientation of the acetabular component. Computer-assisted surgical navigation techniques have been developed but are expensive, invasive, and time-consuming. As such, surgeons more often rely on estimations based on visual landmarks or mechanical guides.

U.S. Pat. No. 8,267,938 describes an instrument that comprises a tripod having an angularly adjustable guide rod on it. The tips of the legs are set on a bone surface of the subject to define a reference plane, and the guide rod is set by the surgeon to a defined orientation with respect to this plane. The guide rod provides the desired orientation for insertion of the acetabular component. The positioning of the legs is determined by preoperative calculations based on subject-specific data, determined for example from computed tomography (CT) studies, or statistical shape models fit to biplanar radiographs. Commercial versions of the described instrument typically require these determinations to be carried out in advance by a third party company and can, therefore, be time consuming and expensive, and by design is invasive. There is also no method for verifying the plan intraoperatively.

U.S. Pat. No. 6,214,014 describes a system for intraoperative positioning of an acetabular component in inclination. The system comprises a goniometer, a laser pointer, and an acetabular insertion handle. In use, the goniometer is positioned adjacent to the teardrop and the superior rim of an acetabular socket. A swing arm of the goniometer is then adjusted for the desired offset and the position is marked on the wall using the laser pointer. After the appropriate mark is indicated on the wall, the goniometer and laser pointer are removed and the prosthetic acetabular cup is inserted with the aid of the handle. The handle is appropriately aligned by inserting the laser pointer and moving the handle until the laser light of the laser pointer is aligned with the previously indicated mark on the wall. Angular alignment for positioning of the acetabular component, therefore, depends on the surgeon's ability to align a mark with the laser pointer. Such a method can be unreliable and susceptible to movement of the pelvis between making the mark and aligning the mark with the laser pointer.

Achieving the correct depth of the acetabular component is also a challenge. Positioning the component at the incorrect depth can lead to loosening due to lack of bone ingrowth, and to changes in leg length and femoral offset due to lateralizing the hip centre. Current methods for determining the depth positioning of an acetabular component rely on visual or auditory cues that are intuitively assessed. For example, surgeons typically rely on the ability to visually gauge depth positioning by observing the bone surface through holes in the implant. Alternatively, surgeons will rely on a change in the sound of the hammer during surgery. These methods are problematic in that visualization is often difficult in the case of obese patients or minimally-invasive surgery, some acetabular components/cups have no holes for visualization, and detecting the correct change in sound depends on surgeon experience. Accordingly, there is considerable uncertainty regarding whether or not the desired depth has been attained, and errors in depth are usually only discovered in the postoperative X-ray.

There continues to be a need for a system and method for positioning an acetabular component in total hip arthroplasty that is universal in design but allows for patient-specific alignment and that is simple, intuitive and accurate to use.

This background information is provided for the purpose of making known information believed by the applicant to be of possible relevance to the present disclosure. No admission is necessarily intended, nor should be construed, that any of the preceding information constitutes prior art against the present disclosure.

### SUMMARY OF THE INVENTION

Disclosed herein are exemplary embodiments pertaining to apparatus, methods, and systems for positioning an acetabular component during hip arthroplasty procedures. An exemplary embodiment of the present disclosure relates to an apparatus for positioning an acetabular component during a hip arthroplasty procedure. The apparatus comprises a positioning member for engaging an acetabular socket. The positioning member has a landing surface for engaging said acetabular socket relative to at least one bone landmark. An elongate guiding member is coupleable to said positioning member about perpendicular to said landing surface. The guiding member is adjustable to a positioning angle setting and is configured to receive a position guide, wherein adjustment of said guiding member to said positioning angle setting orients said position guide onto a target site at said acetabular socket.

In accordance with another aspect of the disclosure, there is provided a method for positioning an acetabular component in hip arthroplasty, the method comprising a) determining a positioning angle from a radiographic image of the subject's pelvis, said positioning angle determined relative to predefined landmarks at the acetabular socket of the pelvis; and b) positioning a position guide at said acetabular socket relative to said landmarks, said position of said position guide corresponding to said positioning angle, whereby said position guide is used to guide the positioning of said acetabular component for implantation in said subject.

In accordance with another aspect of the disclosure, there is provided a method for positioning an acetabular component for implantation during a hip arthroplasty procedure performed on a subject, the method comprising a) determining a positioning angle from a radiographic image of the subject's pelvis, said positioning angle determined relative to predefined landmarks at the acetabular socket of the pelvis; b) positioning a first position guide at said acetabular socket relative to said landmarks, said position of said first position guide corresponding to said positioning angle; c) positioning a second position guide at said acetabular socket relative to said first position guide; whereby said second position guide is used to guide the positioning of said acetabular component for implantation in said subject.

In accordance with another aspect of the disclosure, there are provided methods for verifying the positioning of an acetabular component wherein the positioning is determined according to methods of the instant application. An exemplary embodiment of the present disclosure relates to a device for evaluating the positioning of an acetabular component according to methods of the instant application. The device comprises a) a spherical component having an elongate handle extending therefrom; and b) a marking tool adapted for tracing the acetabular rim of said socket onto the surface of the spherical component when said spherical component is in the position determined according to methods of the present application.

In accordance with a further aspect of the disclosure, there is provided a method for evaluating the positioning of an acetabular component, said positioning determined according to the methods of the present application, the method comprising a) positioning the spherical component of the device according to embodiments of the present application in said acetabular socket relative to said position guide; b) tracing the acetabular rim of said socket onto the surface of the spherical component; and c) evaluating the positioning of said spherical component relative to said traced acetabular rim.

In accordance with another aspect of the disclosure, there is provided a device for evaluating the reamed depth of an acetabular socket, the device comprising two slidably interengaging parts, each part comprising at one end an extension for engaging with a respective landmark at said acetabular socket, wherein said interengaging parts together function as a protractor for determining an angle when said extensions are engaged with said landmarks and said device is positioned in said acetabular socket.

In accordance with a further aspect of the disclosure, there is provided a method for evaluating the reamed depth of an acetabular socket, the method comprising a) determining on a radiographic image a desired location for an acetabular component in said acetabular socket; b) calculating an expected angle on said image; c) positioning the reamed depth evaluation device of the instant application in said acetabular socket to determine an actual angle of the acetabular socket, wherein the extensions of said device are in contact with said landmarks; and d) comparing the actual angle to the expected angle to verify the reamed depth of said acetabular socket.

In accordance with another aspect of the disclosure, there is provided a device for guiding depth positioning of an acetabular component, the device comprising a depth gauge having a calibrated scale for aligning an implant inserter to a desired depth of insertion in the acetabular socket of a subject, wherein the depth gauge is attachable to a position guide.

In accordance with a further aspect of the disclosure, there is provided a kit for positioning an acetabular component during a hip arthroplasty procedure, the kit comprising the apparatus described according to the present disclosure.

### BRIEF DESCRIPTION OF THE DRAWINGS

These and other features of the invention will become more apparent in the following detailed description in which reference is made to the appended drawings.

FIG. 1 is a perspective view of an alignment guide, according to embodiments of the present disclosure;

FIG. 2(a) is a top view of an alignment guide set for right-side positioning, according to embodiments of the present disclosure;

FIG. 2(b) is a top view of an alignment guide set for left-side positioning, according to embodiments of the present disclosure;

FIG. 3 is an end view of an alignment guide, according to embodiments of the present disclosure;

FIGS. 4(a) and 4(b) are perspective views of an alignment guide further comprising an elongated handle and a guiding member for inclination positioning, according to embodiments of the present disclosure;

FIGS. 5(a) and 5(b) are perspective views of an alignment guide further comprising an elongated handle and a guiding member for anteversion positioning, according to embodiments of the present disclosure;

FIGS. 6(a) and 6(b) are views of guiding members comprising keyways for anteversion (V) and inclination (I) positioning respectively, according to embodiments of the present disclosure;

FIG. 6(c) is a side view of an alignment guide comprising a corresponding keyway for insertion of a guiding member, according to embodiments of the present disclosure;

FIG. 7 is a perspective view of an alignment guide with a position guide being set in the inclination orientation, according to embodiments of the present disclosure;

FIG. 8 is a perspective view of an alignment guide with a position guide being set in the anteversion orientation, according to embodiments of the present disclosure;

FIG. 9 is a perspective view of an alignment guide with a first position guide being set in the anteversion orientation and a second position guide being positioned in reference to the first, according to embodiments of the present disclosure;

FIG. 10 is a perspective view of a continuously adjustable alignment guide, according to embodiments of the present disclosure;

FIG. 11 is a perspective view of a continuously adjustable alignment guide including a handle and a position guide, according to embodiments of the present disclosure;

FIG. 12 is a side view of a continuously adjustable alignment guide in rotation, according to embodiments of the present disclosure.

FIG. 13 is a perspective view of a continuously adjustable alignment guide, according to embodiments of the present disclosure;

FIG. 14 is a perspective view of a double-barreled alignment guide with a first position guide being set in the anteversion orientation and a second position guide being positioned in reference to the first with the assistance of an alignment indicator member, according to embodiments of the present disclosure;

FIG. 15 is a side perspective view of a double-barreled alignment guide, further comprising an elongated handle and a guiding member for anteversion positioning, according to embodiments of the present disclosure;

FIG. 16 is a side perspective view of an implant inserter aligned with a position guide positioned at an acetabular socket, according to embodiments of the present disclosure;

FIGS. 17 (a), (b), (c) are side perspective views of trial and final implant inserters showing alignment of a depth gauge, according to embodiments of the present disclosure;

FIG. 18 is a side perspective view of an implant inserter aligned with a position guide using an alignment guide, according to embodiments of the present disclosure;

FIGS. 19 (a) and (b) are side perspective views of a position evaluation device, according to embodiments of the present disclosure;

FIG. 20 (a) is a perspective view of a depth verification device, according to embodiments of the present disclosure, FIG. 20 (b) is a perspective view of the depth verification device shown in Figure (a) positioned in an acetabular socket, and FIG. 20 (c) is a view of the X-ray verification procedure, according to embodiments of the present disclosure;

FIGS. 21 (a), (b), (c) are views of the 3D templating procedure for determining inclination and anteversion, according to embodiments of the present disclosure. FIG. 21(a) is an image of the computed tomography (CT) slice parallel to the APP to measure the inclination, according to embodiments of the present disclosure; FIG. 21(b) is an image of the CT slice perpendicular to the APP to measure anteversion, according to embodiments of the present disclosure; FIG. 21(c) is an image of a slice through a segmented CT scan, showing the 3D APP, according to embodiments of the present disclosure;

FIGS. 22 (a) and (b) are views of the anteroposterior (AP) radiograph templating procedure used for determining inclination alone, according to embodiments of the present disclosure;

FIG. 23 is a side perspective view of a position evaluation device, according to embodiments of the present disclosure;

FIG. 24 is a perspective view of the interior of the position evaluation device shown in FIG. 22, absent the handle component, according to embodiments of the present disclosure;

FIGS. 25 (a) and (b) are perspective views of crosshairs, according to embodiments of the present disclosure; and

FIGS. 26 (a), (b), and (c) are perspective views illustrating the positioning of a crosshair on the acetabular rim such that the T-marked leg is placed on the teardrop and the N-marked leg is placed on the anterior notch of the acetabulum.

### DETAILED DESCRIPTION OF THE INVENTION

### Definitions

The term “anteversion” as used herein refers to the degree of tilt of the axis of the acetabular component towards the front of the subject relative to the anterior pelvic plane (which is usually roughly perpendicular to the transverse plane). The industry accepted range of anteversion is about 5° to about 25°, and is referred to as the “safe zone”.

The term “inclination” as used herein refers to the degree of tilt of the axis of the acetabular component upward relative to the anterior pelvic plane (which usually roughly corresponds to the coronal plane). The industry accepted range of inclination is about 30° to about 50°, and is referred to as the “safe zone”.

The term “subject” as used herein refers to a mammalian patient in need of total hip arthroplasty. Mammalian patients are exemplified by humans, primates, equines, ruminants, felines, canines, and the like.

As used herein, the term “about” refers to a variation within the range of about plus 10% to about minus 10% from the nominal value. It is to be understood that such a variation is always included in any given value provided herein, whether or not it is specifically referred to.

Unless defined otherwise, all technical and scientific terms used herein have the same meaning as commonly understood by one of ordinary skill in the art to which this disclosure belongs.

The apparatus and method according to the present disclosure provide for simple and intuitive determination of a visual reference for positioning of an acetabular component in hip arthroplasty. The method, according to embodiments of the disclosure, allows the surgeon to quickly and easily determine patient specific acetabular orientation preoperatively, thereby increasing the accuracy of the component placement and potentially reducing the surgical time for the patient. Methods according to embodiments of the disclosure, further rely on position references that are established relative to the bone itself, ensuring accuracy particularly in normally challenging cases, for example, in minimally invasive surgeries, obese patients, and even if the patient moves during surgery.

The apparatus according to the present disclosure comprises a minimal number of cooperating parts for positioning an acetabular component in hip arthroplasty. The simplicity in design facilitates cost effective manufacture and facility in cleaning for reuse or for disposable use. In some embodiments, the system can be manufactured for disposable use with modest manufacturing costs. The apparatus of the present disclosure is a universal device that allows for patient-specific alignment. Since only a guide pin is used, the apparatus of the present disclosure allows the surgeon flexibility, in cases where alternate placement is indicated during surgery. Moreover, the apparatus and methods of the present disclosure can be easily adopted into current surgical workflow practices with minimal change to surgical workflow.

The apparatus and methods according to the present disclosure allow positioning of an acetabular component to be determined based on the inclination orientation, the anteversion orientation, or a combination of both the inclination and anteversion orientation. In further embodiments, the apparatus and methods according to the present disclosure allow positioning of an acetabular component to be determined based on the angular orientation, as discussed above, as well as by the depth positioning. In such embodiments, therefore, the positioning of an acetabular component can be guided by a combination of any one or more of inclination orientation, anteversion orientation, a combination of both inclination and anteversion orientation, and depth. In this way, the apparatus and method according to the present disclosure provides further flexibility to the surgeon.

Quick and easy evaluation and verification of the position of the acetabular component, determined using the apparatus and methods of the present disclosure, is further made possible by verification tools and methods according to the present disclosure. The surgeon can, thereby, easily confirm the determined positioning of the acetabular component without disruption to the surgical procedure.

The apparatus of the present disclosure can be provided in a kit to facilitate usage. Specifically, one or more components of the apparatus and/or one or more of the devices described herein can be provided in a kit for the particular desired arthroplasty procedure.

### Apparatus—Alignment Guide

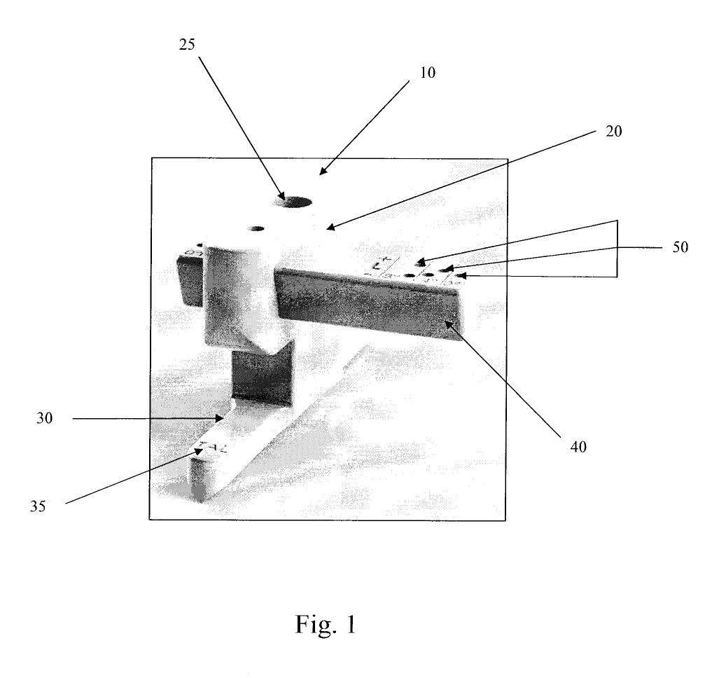

Referring now to the drawings, in which like reference numerals identify identical or substantially similar parts throughout the several views, FIG. 1 illustrates a perspective view of an alignment guide 10 according to embodiments of the present disclosure. Alignment guide 10 includes a positioning member 20 adapted for positioning adjacent to an acetabular socket. In some embodiments, the positioning member 20 can be made adaptable at one end for optional connection to an elongate handle. The handle may be aligned with or offset from the main axis of insertion. In such embodiments, it is contemplated that the positioning member 20 can be interchangeable with other surgical instruments on a standard surgical handle.

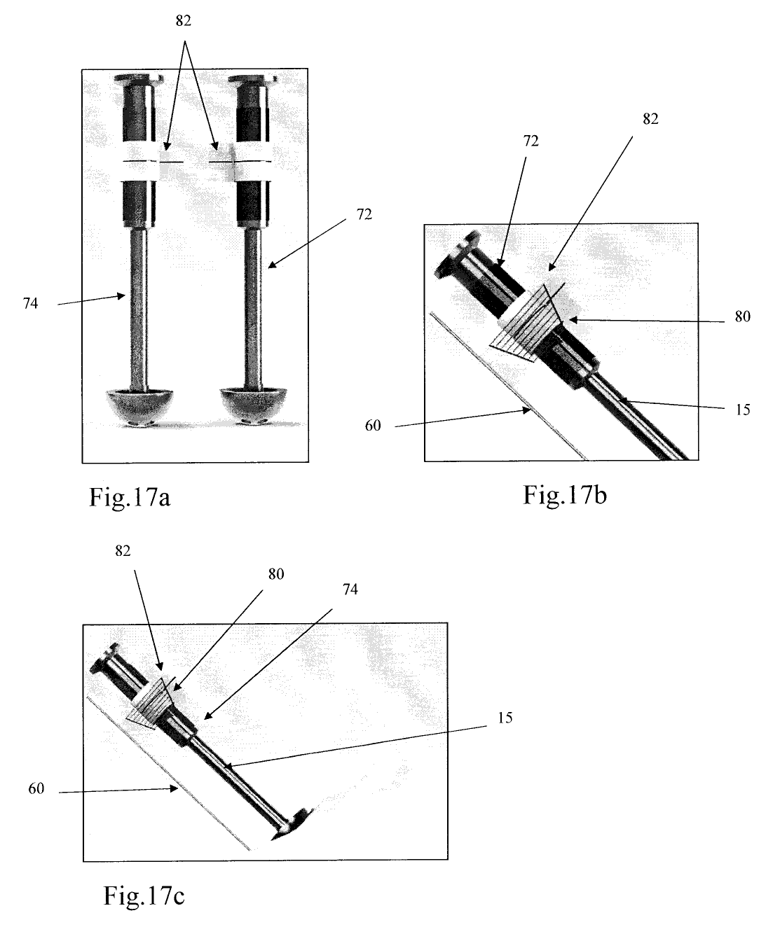

The positioning member 20 can comprise at least one handle coupling 25 for receiving the elongate handle. As shown in FIG. 1, some embodiments of the positioning member 20 comprise a single handle coupling 25 for attaching the positioning member 20 onto the handle 15. In other embodiments, alternate positions of the handle 15 are made available to the surgeon. As shown in FIG. 15, for example, the positioning member 20 may comprise two handle couplings 25. In one embodiment, the positioning member 20 comprises a left offset and a right offset handle coupling 25. In this way, the handle 15 may be coupled to the positioning member 20 offset on either the left or right side of the positioning member 20 in accordance with the surgeon's preference or to accommodate the placement of additional surgical instruments at the acetabulum, for example a drill.

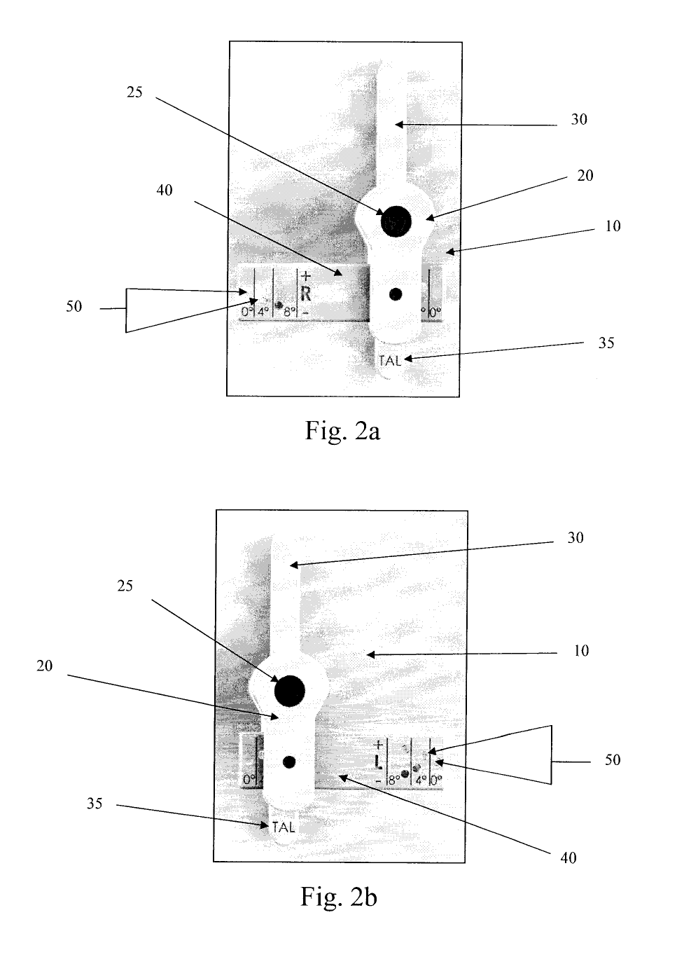

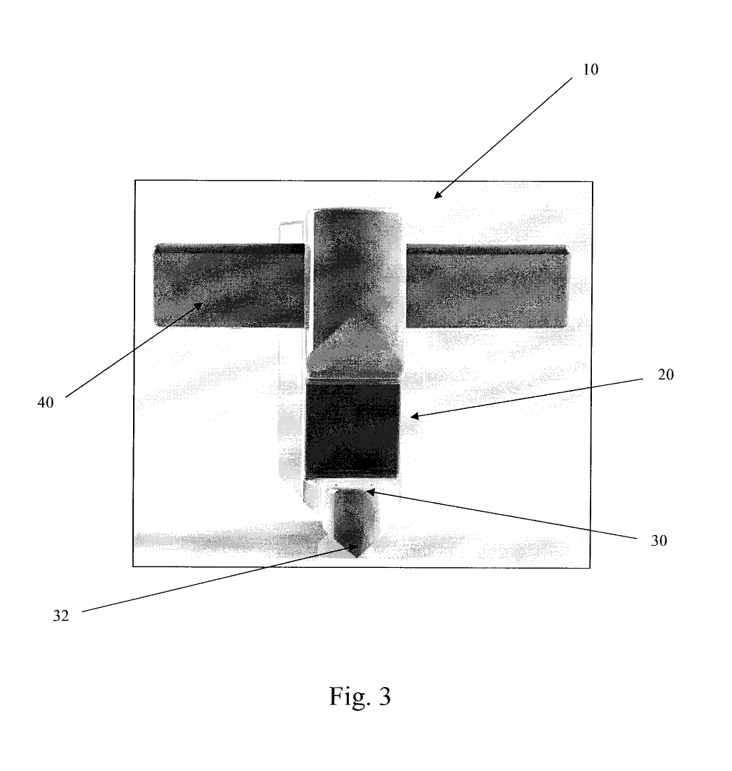

At a second end, the positioning member 20 has a landing surface 30 adapted for positioning adjacent to the acetabular socket. The landing surface 30 is adapted for positioning on the acetabular socket relative to at least one bone landmark. In some embodiments, as shown in FIGS. 1 and 15, the positioning member 20 may comprise orientation indicia 35 to provide a visual reference for ensuring that the landing surface 30 is oriented in the desired direction at the acetabulum. For example, as shown in FIGS. 2(a) and (b), indicia “TAL” 35 are used to indicate the general direction of the tranverse acetabular ligament. In other embodiments, the orientation indicia may comprise an arrow shape 35 on the top surface of the positioning member 20 to indicate the general superior direction (FIG. 15).

In some embodiments, the alignment guide 10 is designed to fit underneath the transacetabular ligament (TAL) in order to access the bone landmarks at the acetabular socket. In other embodiments, the TAL may be removed to access the bone landmarks depending on the surgeon's preference. To accommodate the TAL, the landing surface 30 according to some embodiments of the present disclosure, may have a height of from about 5 mm to about 10 mm. In other embodiments, the height of the landing surface 30 may range from about 5 mm to about 8 mm. In a further embodiment, the height of the landing surface is about 8 mm.

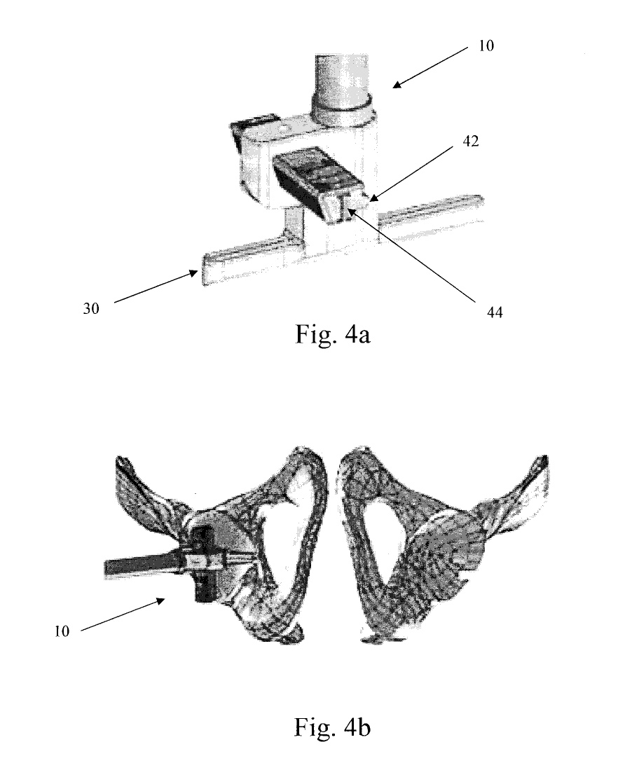

The bone surface at the acetabular socket can be uneven and difficult for stably engaging the landing surface 30 when positioning the alignment guide 10 at the target site of the acetabular socket. According to embodiments of the present disclosure, the landing surface 30 can be adapted to facilitate positioning at the target site. For example, the landing surface 30, in some embodiments, can be rounded in shape. The rounded shape of the landing surface 30 allows the landing surface 30 to be tilted in a controlled and predictable manner for determining the orientation angle. In a preferred embodiment, as shown in FIG. 3, the landing surface 30 is adapted to be V-shaped 32 such that the peak of the V-shaped surface 32 engages the target site at the acetabular socket relative to the bone landmark(s). In this way, the landing surface 30 can be more securely positioned at the target site. The V-shaped landing surface 30, according to this embodiment, can be aligned with marks made during the surgery at the appropriate locations to allow the landing locations to be precisely defined. The line contact achieved with the V-shape further allows for predictable tilting about the previously defined line, thereby, allowing the device to be tilted easily in order to simultaneously achieve the desired inclination and anteversion orientations. The landing surface 30 ranges in length in order to accommodate a variety of acetabular socket sizes. Typically the mean diameter of a normal human adult acetabulum ranges from about 43 mm to 57 mm. The length of the landing surface, in embodiments suitable for use in human adult subjects, is generally longer than the diameter of the acetabulum to allow for translation across the rim surface for finding an optimal location for placement of the position guide 60. For example, the landing surface 30 can range from 40 to 80 mm in length. In one embodiment, the landing surface 30 ranges from 40 to 50 mm in length. In another embodiment, the landing surface 30 ranges from 50 to 60 mm in length. In further embodiments, the landing surface 30 ranges from 60 to 70 mm in length. In other embodiments, the landing surface 30 ranges from 67 to 77 mm in length.

An elongate guiding member 40 is coupled to the positioning member 20. In some embodiments, the guiding member 40 is coupled about perpendicular to the outwardly extending landing surface 30 of the positioning member 20. The guiding member 40 is configured to set the alignment guide 10 at the desired positioning angle.

### a) Positioning Angle Setting—Continuously Adjustable

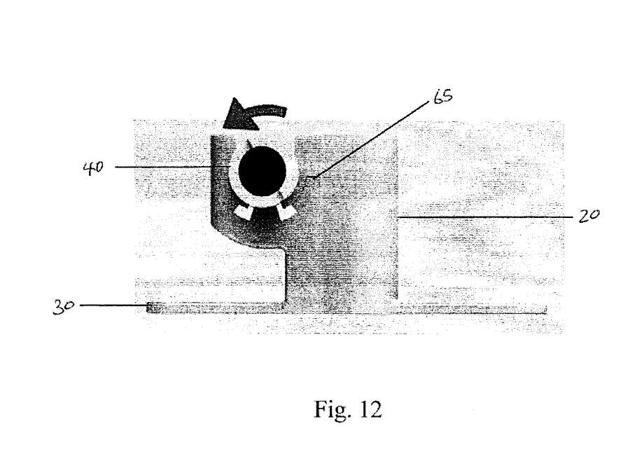

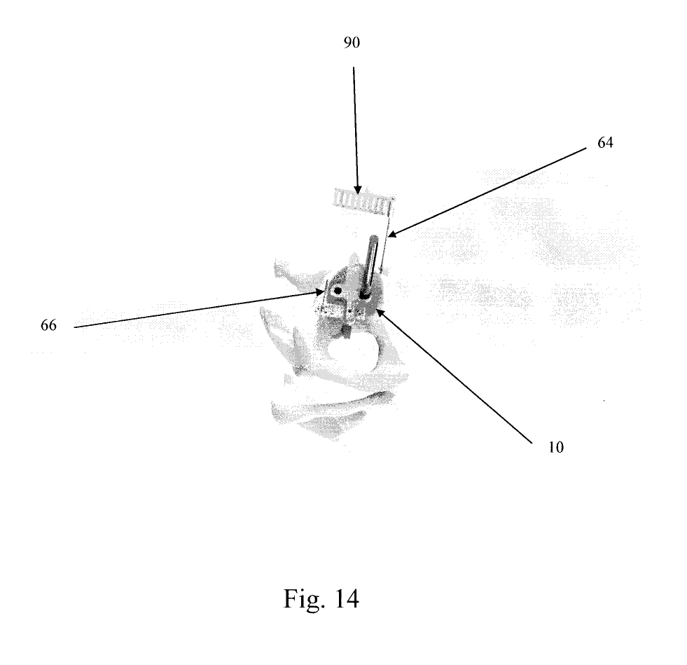

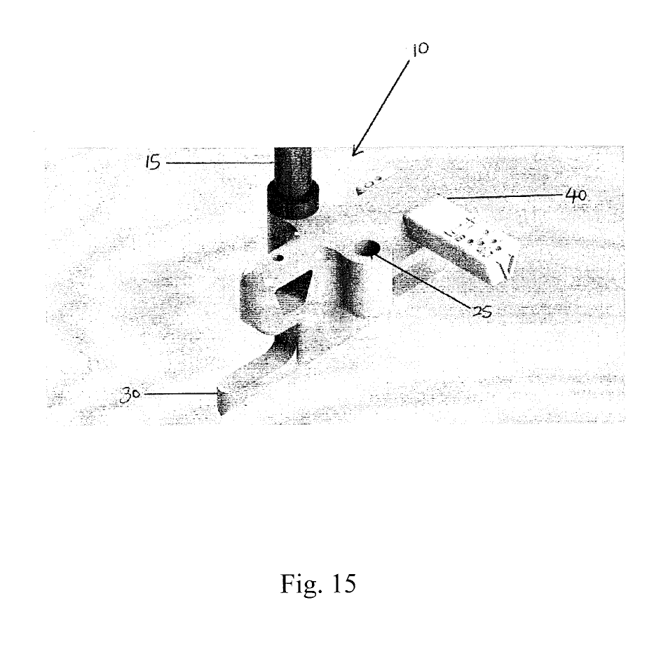

In some embodiments, the guiding member 40 is continuously adjustable, for example, rotatably adjustable to a desired positioning angle. As shown in FIGS. 10-13, for example, the guiding member 40 is rotatable relative to the positioning member 20 to permit setting at the positioning angle setting. According to this embodiment, the guiding member 40 can include means for controlling the degree of rotation of the guiding member 40 relative to the positioning member 20. In this way, the guiding member 40 can be rotated and set to the predetermined positioning angle setting. In some embodiments, as shown in FIG. 10, the guiding member 40 displays teeth 121 that cooperatively engage with corresponding teeth (not shown) presented on the positioning member 20. As shown in FIG. 12, the guiding member 40 rotates within a track 65 set in the positioning member 20. In other embodiments, the guiding member 40 includes a dial 61 for controlling the rotation of the guiding member 40 relative to the positioning member 20 (FIG. 13).

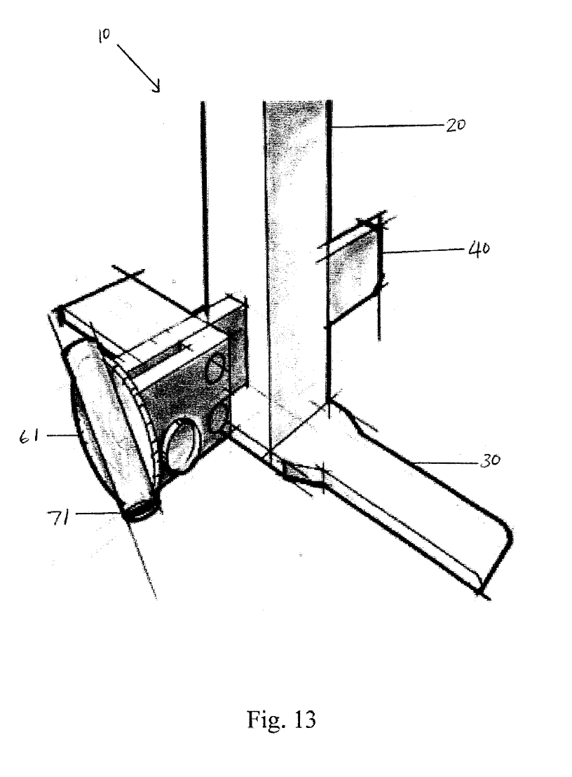

As shown in FIG. 11, the alignment guide 10 is adapted to be couplable to a position guide 60 to allow the coupled position guide 60 to be oriented in alignment with the predetermined positioning angle and released from the alignment guide 10 once fixed into position in the pelvic bone of the patient. As shown in the embodiment illustrated in FIGS. 10, 11, and 12, the position guide 60 is releasably coupled to the guiding member 40 by way of grooves 75 that allow the position guide 60 to be held in place. In other embodiments (FIG. 13), the guiding member 40 includes a sheath 71 through which the position guide 60 is positioned and held in place. The position guide 60 can take a variety of forms that will be apparent to those skilled in the art, for example, the position guide 60 can be a guidewire or a bone pin.

In some embodiments of the disclosure, the alignment guide 10 is adapted to allow slidable translation of the guiding member 40 in an about perpendicular direction to the positioning member 20. For example, the positioning member 20 can be slidingly translated relative to the geometry of the acetabular socket of the particular patient. In this way, the positioning member 20 can be adjusted to accommodate different acetabular shapes and sizes. FIG. 13 shows one embodiment of a guiding member 40 in slidable engagement with the positioning member 20. In this embodiment, the guiding member 40 is slidable through the positioning member 20 to permit slidable translational movement.

### b) Positioning Angle Setting—Fixed Settings

In other embodiments, as shown in FIG. 1, the guiding member 40 is configured to at least one fixed setting. In such embodiments, the guiding member 40 comprises at least one, and preferably a plurality of, positioning openings 50 along the length of the guiding member 40, each sized to receive a position guide 60. Each respective positioning opening 50 corresponds to a predetermined positioning angle setting such that a position guide 60, when coupled to the guiding member 40 at a positioning opening 50, is oriented relative to the acetabular socket at the set positioning angle.

In some embodiments, the guiding member 40 comprises a plurality of positioning openings 50 corresponding to multiple fixed positioning angles. In this way, the guiding member 40 offers a range of positioning angle settings that can be selected without requiring any adjustment to the guiding member 40 itself, thus minimizing the introduction of human error in setting the positioning angle, avoiding accidental adjustment of the angle setting during use, and making finer increments available. In this way, the fixed positioning openings 50 facilitate accurate positioning of the position guide 60 at the desired positioning angle. In one embodiment, the guiding member 40 comprises a plurality of positioning openings 50 corresponding to multiple positioning angle settings fixed at increasing and/or decreasing increments. In one embodiment, the positioning openings 50 are oriented on the guiding member 40 such that the openings point toward a common axis. For example, as illustrated in FIGS. 2(a) and 2(b), the openings 50 are aligned in a “V” shape, as this geometry makes it easiest to target the pin into the ischial notch.

In certain embodiments, the positioning openings 50 correspond to multiple positioning angle settings fixed at increasing and/or decreasing increments ranging from about 1° to about 5°. In another embodiment, the positioning openings 50 correspond to multiple positioning angle settings fixed at increasing and/or decreasing increments of about 5°. In a further embodiment, the multiple positioning angle settings are fixed at increasing and/or decreasing increments of about 4°. In another embodiment, the multiple positioning angle settings are fixed at increasing and/or decreasing increments of about 3°. In a further embodiment, the multiple positioning angle settings are fixed at increasing and/or decreasing increments of about 2°. In a preferred embodiment, the multiple positioning angle settings are fixed at increasing and/or decreasing increments of about 1°. In certain embodiments, the range of incremental positioning angle settings is achieved with multiple interchangeable guiding members 50 as discussed in more detail below.

In some embodiments of the disclosure, the guiding member 40 slidably translates along an about perpendicular direction to the positioning member 20. FIGS. 1 and 15 show embodiments of a guiding member 40 in slidable engagement with the positioning member 20. In these embodiments, the guiding member 40 is slidable through the positioning member 20 to permit slidable translational movement. The slidable translation of the guiding member 40 provides versatility of the alignment guide 10. In some embodiments, the guiding member 40 slidingly engages with the positioning member 20 by friction fit. In other embodiments, the guiding member 40 slidingly engages with the positioning member 20 and is further fixed in place with a releasable fastener, for example a set screw.

Further versatility is afforded to the surgeon (shown in FIGS. 2(a) and 2(b), for example) as the slidable translation of the guiding member 40 allows the alignment guide 10 to be adapted for use on either the right or left hip of a patient. In this embodiment, the guiding member 40 comprises a plurality of positioning openings 50 at both of its opposing ends for use on the right and left hip, respectively.

Slidable translation of the guiding member 40 further provides for interchangeability of the guiding member 40. The alignment guide 10, according to some embodiments of the present disclosure, may be supplied as a kit with a plurality of guiding members 40 each offering a different range of positioning openings 50 to choose from. In operation, a guiding member 40 comprising the desired range of positioning openings 50 can be selected and slidably interchanged and/or coupled to the positioning member 20. For example, in one embodiment, multiple interchangeable guiding members 40 are available to choose from, each comprising a different range of positioning openings. In this way, the guiding member 40 comprising the most appropriate range of positioning openings that correspond most closely (e.g., within 1°) to the desired positioning angle may be selected. In one embodiment, the guiding member 40 comprises positioning openings corresponding to positioning angles increasing and decreasing by increments of 4° and ranging from −8° to +8° (FIG. 2). For example, the positioning openings may correspond to positioning angles −8/−4/0/4/8°. In another embodiment, the guiding member 40 comprises positioning openings corresponding to positioning angles increasing and decreasing by increments of 4° and ranging from −9° to +9°. For example, the positioning openings may correspond to positioning angles −9/−5/−1/1/5/9°. In a further embodiment, the guiding member 40 comprises positioning openings corresponding to positioning angles increasing and decreasing by increments of 4° and ranging from −10° to +10°. For example, the positioning openings may correspond to positioning angles −10/−6/−2/2/6/10°. In a further embodiment, the guiding member 40 comprises positioning openings corresponding to positioning angles increasing and decreasing by increments of 4° and ranging from −11 to +11°. For example, the positioning openings may correspond to positioning angles −11/−7/−3/3/7/11°. In certain embodiments, a series of interchangeable guiding members 40 may be provided to cover a range of positioning angles. In one embodiment, for example, the series of guiding members 40 together provide a range of positioning angles from −11 to 11 in 1° increments. In another embodiment, the series of guiding members 40 together provide a range of positioning angles from −10 to 10 in 2° increments.

In operation, the maneuverability of surgical instruments within the acetabulum can be limited and may make it challenging to accurately position a position guide 40 at a desired location in the acetabulum. As shown in FIG. 1, an embodiment of the alignment guide 10 can have a single-barreled positioning member 20 in order to minimize the size of the device and faciliate its use in the limited space of the surgical site. Other embodiments of the alignment guide 10, as shown in FIG. 15, have a double-barreled positioning member 20, thereby, allowing the guiding member 40 to be coupled to it in more than one position and in this way offer improved maneuverability of the alignment guide 10. Referring to FIG. 15, for example, the positioning guide 20 can be configured to slidingly receive a guiding member 40 in one of two opposing locations. In one embodiment, the positioning guide 20 is configured to slidingly receive a guiding member 40 in more than one location. In a further embodiment, the positioning guide 20 is configured to slidingly receive a guiding member 40 in one of two locations. In other embodiments, as shown in FIG. 1, the positioning guide 20 is configured to slidingly receive a guiding member 40 in a single location.

In a further embodiment, the guiding member 40 can be slidingly translated relative to the geometry of the acetabular socket of the particular patient. In this way, the guiding member 40 can be adjusted to accommodate different acetabular shapes and sizes.

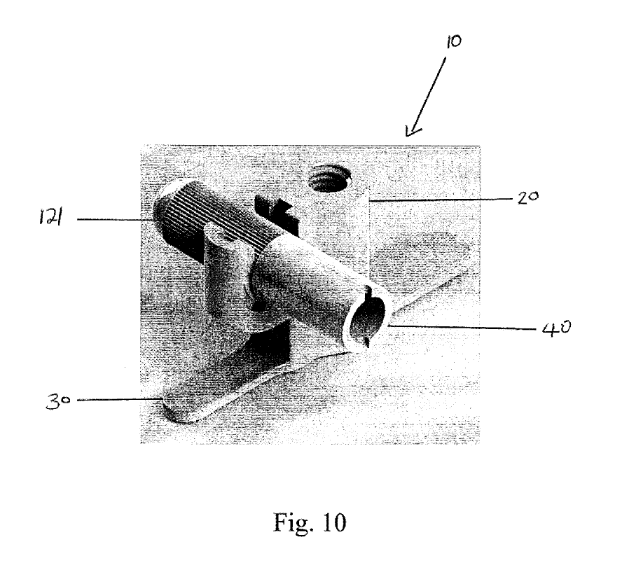

Referring to FIG. 8, for example, the alignment guide 10 is adapted to be couplable to a position guide 60 to allow the coupled position guide 60 to be oriented in alignment with the predetermined positioning angle and released from the alignment guide 10 once fixed into position in the pelvic bone of the patient. As shown in the embodiment illustrated in FIG. 1, the positioning openings 50 are sized to firmly retain the position guide 60 in place when inserted therein and to be slidably released from the positioning opening 50 when required. The position guide 60 can take a variety of forms that will be apparent to those skilled in the art, for example, the position guide 60 can be a bone pin (also called a K-wire). In certain embodiments, the position guide 60 can further comprise a support to facilitate the accuracy of the bone pin entering the bone at the intended angle. For example, in embodiments wherein the position guide 60 is a bone pin, the bone pin may further include a supporting sheath and the positioning openings sized to accommodate same.

### Preoperative Orientation

In operation, the alignment guide 10, of the present disclosure, is set to the desired positioning angle for determining the positioning orientation of the acetabular component. It is contemplated that any method for determining the positioning angle may be used by those of skill in the art and is not limited to those methods described herein.

The methods according to embodiments described herein, allow the surgeon to quickly and easily determine patient specific acetabular orientation preoperatively based on position references that are established relative to the patient's bone itself. The methods determine a positioning angle specific to the patient based on preoperative radiographic templating.

The apparatus and methods according to the present disclosure allow positioning of an acetabular component to be determined based on the inclination orientation, the anteversion orientation, or a combination of both the inclination and anteversion orientation using a single device and requiring only slight modifications.

### Radiographic Templating

### a) Anteroposterior (AP) Radiographic Templating

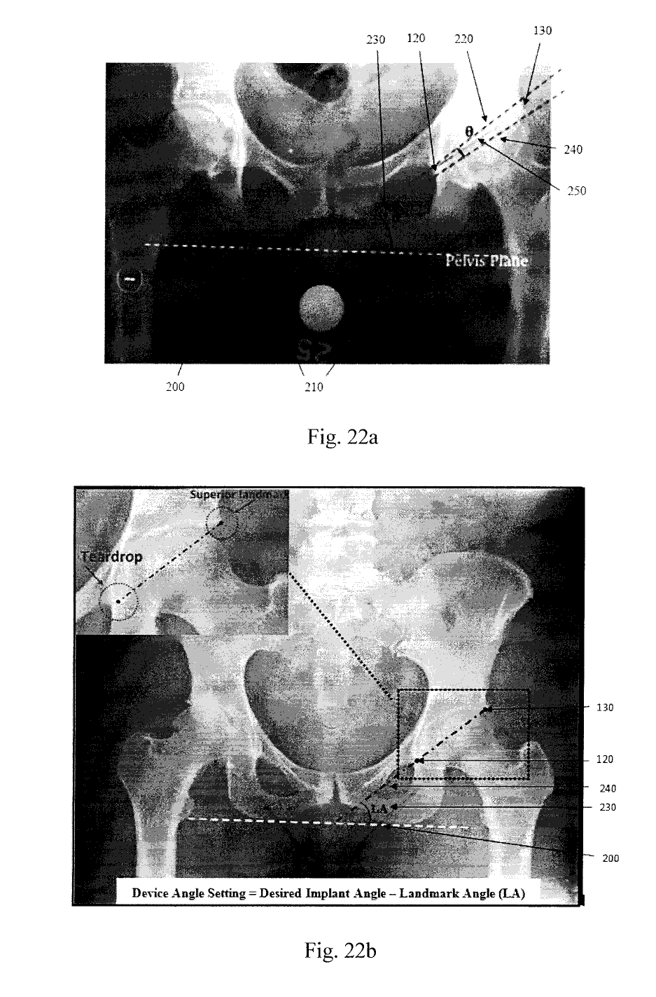

The templating procedure for determining inclination orientation can be determined from anteroposterior (AP) radiographs of the pelvis of the subject. In one embodiment, the positioning angle is determined from a single anteroposterior (AP) radiograph. In this way, the AP radiograph is a useful radiographic template for determining the positioning angle. As illustrated in FIG. 22, the positioning angle is determined by first determining a reference line 200 on the AP radiograph by drawing a horizontal line between a pair of landmarks to define the pelvic plane. In some embodiments, the ischial tuberosities 210 at the bottom of the pelvis are used to define the pelvic plane. In other embodiments, the two teardrops of the acetabular socket are used to define the pelvic plane. Once the reference line 200 is drawn on the AP radiograph, a second line is drawn on the radiograph from the teardrop 120 of the acetabular socket to the opposite superior edge 130 of the acetabular socket, i.e., the teardrop-superior landmark line 240. The teardrop-superior landmark line 240 is then extended down to the reference line 200 to determine the landmark angle “α” or “LA” 230.

A desired implant angle is preselected by the surgeon based on the safe zone and personal preference. In some embodiments, an implant angle of 30°, 35°, 40°, 45°, or 50° relative to the reference line 200 is preselected. In other embodiments, the implant angle is 40° relative to said reference line 200. The landmark angle α or LA 230 is then subtracted from the preselected desired implant angle to determine the positioning angle 250 for setting the alignment guide 10. This calculation can be represented as follows.

Positioning Angle=Desired Implant Angle−Landmark Angle

To illustrate, if the landmark angle 230 is determined from the radiograph as being 36° to the horizontal reference line 200, and the desired implant angle is preselected at 40°, then the positioning angle 250 would be +4°.

### b) 3D Radiographic Templating

In alternative embodiments, where positioning of an acetabular component based on the anteversion orientation or a combination of both the inclination and anteversion orientations is desired, the relevant positioning angles can be determined from 3D radiographic templating, for example a CT scan, or a statistical shape model fit to one or more X-rays. In this case, the anteversion and inclination angles of the natural acetabulum are determined relative to the 3D APP, and again the relative angle of the alignment guide is determined by subtracting the anteversion or inclination angle from the respective desired anteversion or inclination.

### Inclination Orientation

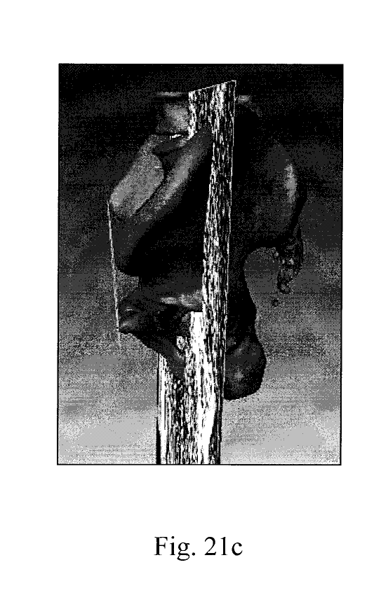

Referring to FIG. 21(a), in a further embodiment the positioning angle 250 for determining the inclination orientation can also be identified from a CT model that has been manipulated such that the slices are made to be parallel to the APP, and by measuring the angle between a line drawn from the acetabular teardrop to the lateral acetabular margin and the interteardrop line.

### Anteversion Orientation

Referring to FIG. 21(b), in one embodiment the positioning angle 250 for determining the anteversion orientation can be identified from a CT image that is perpendicular to the APP (close to the axial plane), and by measuring the angle between a line drawn from the anterior acetabular margin to the posterior acetabular margin and a line perpendicular to the APP.

### c) Marking Landing Position

As described above, the positioning angles are determined based on position references that are established relative to the bone itself. Accordingly, it is desirable to be able to position the alignment guide 10 on the acetabular rim of the patient as accurately as possible, relative to the bone landmarks. In one embodiment, as discussed further below, the landing surface 30 is positioned on two perpendicular planes, the AP plane and the roughly transverse plane perpendicular to the APP, when positioning the position guide(s) 60.

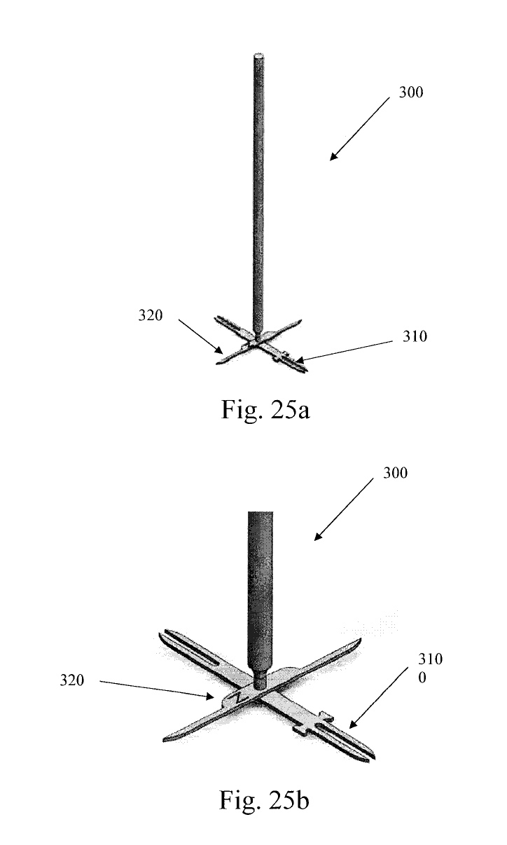

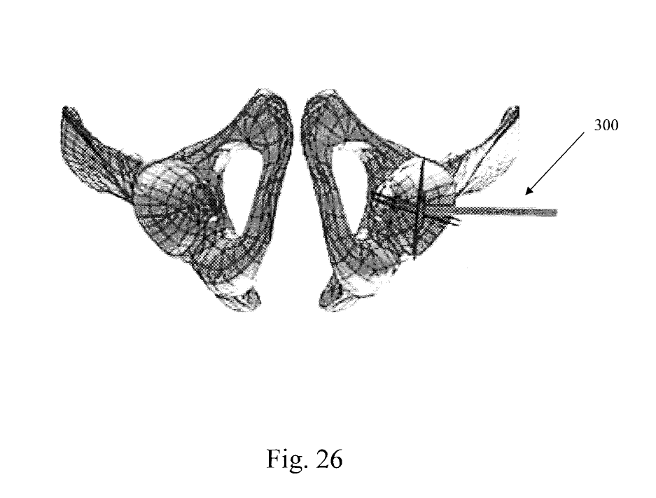

In certain embodiments, markings that correspond to the bone landmarks can be made directly on the acetabular rim of the patient in order to ensure accurate placement of the landing surface 30 of the alignment guide 10. According to one embodiment (FIG. 25), crosshairs 300 can be used to directly mark the position of the bone landmarks on the acetabular rim of the patient. The angle of the crosshairs can first be templated on the 3D model of the pelvis from CT, MRI, or SSM images, for example. Then the surgeon can position the crosshairs on the acetabular rim while keeping the T-marked leg 310 on the teardrop and the N-marked leg 320 on the anterior notch. Then by using a marker or cautery tool, the four bone landmarks are directly marked on the acetabular rim for positioning the alignment guide 10 on the bone landmarks as discussed further below (FIG. 26). In one embodiment, as shown in FIG. 26, the crosshairs comprises four arms to mark the four landmarks. In other embodiments, for example where only a single angular orientation is being determined, e.g., inclination orientation for example, the crosshairs may comprise two arms to mark the landmarks. In certain embodiments, the crosshairs can comprise three arms.

### Setting the Positioning Angle

Once the positioning angle 250 has been determined, the guiding member 40 is adjusted to the corresponding setting. In some embodiments (FIGS. 10 to 13), as already discussed, the guiding member 40 may be rotatably adjusted to the desired positioning angle. In other embodiments, as shown in FIG. 1 for example, the desired positioning angle corresponds to a fixed positioning opening 50 on the guiding member 40 and the position guide 60 is coupled to the selected positioning opening 50 to set it at the desired positioning angle. In this way, the position guide 60 can be quickly and easily positioned at the desired positioning angle without manual adjustment. The alignment guide 10 is then positioned at the acetabular socket of the subject such that the outwardly extending landing surface 30 is placed relative to the corresponding bone landmarks.

Angular orientation of the acetabular component with respect to inclination and/or anteversion can be determined by the positioning of the alignment guide 10 relative to the corresponding bone landmarks. In this way, both orientations can be determined using a single device.

### a) Inclination Orientation

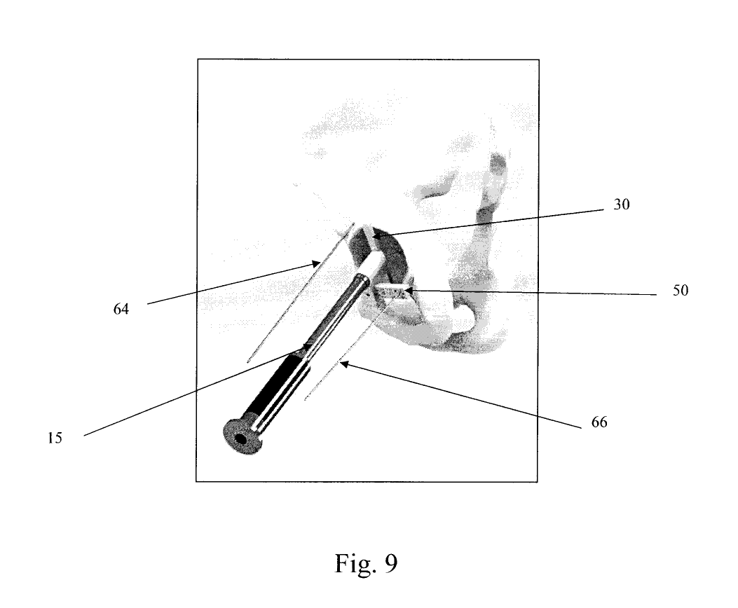

Referring to FIG. 7, the position guide 60 coupled to the selected positioning opening 50 sets the alignment guide 10 at the corresponding positioning angle that was determined by preoperative radiographic templating. When the set alignment guide 10 is positioned at the acetabular socket of the subject such that the landing surface 30 is placed on the corresponding bone landmarks, inclination orientation for the acetabular component is established. To position the landing surface 30 on the bone landmarks, persons of skill in the art will readily appreciate surgical techniques necessary to expose the bone landmark surfaces. For example, removal of the acetabular labrum may be needed.

In one embodiment, the set alignment guide 10 is placed such that one end of the landing surface 30 engages the teardrop 120 of the target acetabular socket and the opposite end of the landing surface 30 engages the superior edge 130 of the acetabular socket. In this way, the landing surface 30 can be said to be directly aligned with the bone landmarks. In other embodiments, the alignment of the landing surface 30 with the bone landmarks is slightly skewed to more closely correspond with the X-ray template. For example, in one embodiment, the alignment of the landing surface 30 is skewed to be offset to the left of the superior 12 o'clock position. In another embodiment, the alignment of the landing surface 30 is skewed to be offset to the right of the superior 12 o'clock position.

Once in position, the position guide 60 is fixed into place and acts as a visual reference for inclination orientation, i.e., the inclination guide 62. Placement of the position guide 60 may depend on the surgical approach taken by the surgeon. In one embodiment, the position guide 60 is positioned within the ischial sulcus of the pelvic bone of the subject (FIG. 7), appropriate for the posterolateral surgical approach for example. In another embodiment, the position guide 60 is positioned within the anterosuperior margin of the acetabular rim, appropriate for the direct lateral (modified Hardinge) or anterolateral surgical approach for example.

In one embodiment, the inclination guide 62 is a bone pin that is drilled into position on the pelvic bone. In this way, the inclination guide 62 is a useful visual reference for angular orientation, specifically inclination, and the alignment guide 10 can then be removed.

### b) Anteversion Orientation

Angular orientation of the acetabular component with respect to anteversion can also be determined based on the positioning angle calculated by radiographic templating simply by repositioning the elongate landing surface 30 relative to the bone landmarks. Referring to the embodiment shown in FIG. 8, the position guide 60 is coupled to the selected positioning opening 50 to set the alignment guide 10 at the corresponding positioning angle. By positioning the elongate landing surface 30 at roughly 90° to the inclination position at the acetabular socket of the subject, anteversion orientation for the acetabular component is established.

In particular embodiments, the set alignment guide 10 is positioned at the acetabular socket of the subject such that the elongate landing surface 30 is placed on the anterior acetabular notch (AAN) at the anterior margin on the rim of the acetabulum and on the opposite posterior margin on the rim of the acetabulum.

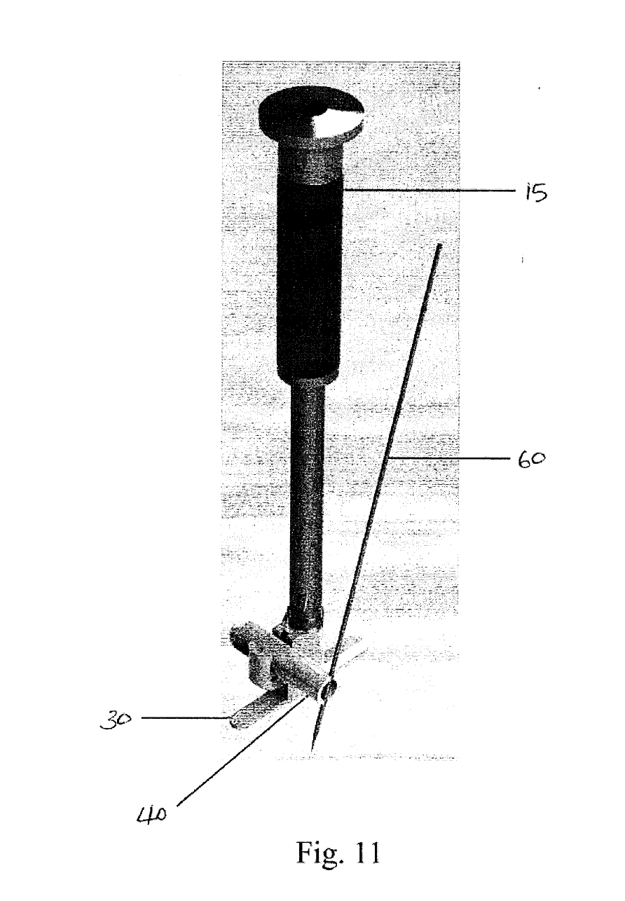

Once in position, the position guide 60 is fixed into place in the pelvic bone of the subject (FIG. 9) and acts as a visual reference for anteversion orientation, i.e., the anteversion guide 64. Placement of the position guide 60 may depend on the surgical approach taken by the surgeon. In one embodiment, the position guide 60 is positioned at the anterosuperior margin of the pelvic bone of the subject (FIG. 7), appropriate for the posterolateral, direct lateral and anterolateral surgical approaches for example.

Once in place, the anteversion guide 64 acts as a visual reference for angular orientation, specifically anteversion, and the alignment guide 10 can then be removed.

In certain embodiments, the inclination guide 62 once in place can be used as a visual guide for determining placement of the anteversion guide 64. For example, once the inclination guide 62 has been positioned, the anteversion guide 64 is coupled to the positioning opening 50 and the landing surface 30 is repositioned to engage the bone landmarks at approximately 90° to the inclination positioning of the landing surface 30. The position of the alignment guide 10 is then adjusted in reference to the inclination guide 62 by tilting the handle 15 to align with the inclination guide 62. In this way, the placement of the anteversion guide 64 is determined.

### c) Combined Inclination/Anteversion Orientation

In further embodiments, the angular orientation of the acetabular component with respect to both inclination and anteversion can be determined for positioning an acetabular component. Referring to the embodiment shown in FIG. 9, the anteversion guide 64 once in place, can operate as a visual guide for determining placement of a second position guide 60, i.e., the combined guide 66. Once the combined guide 66 is in place, the alignment guide 10, and optionally the anteversion guide 64, may be removed. The combined guide 66 is then in position to act as a visual reference for angular orientation that accounts for both inclination and anteversion.

According to such embodiments, the positioning of the combined guide 66 is determined by reference to the anterversion guide 64. Once the anteversion guide 64 has been positioned, the combined guide 66 is coupled to the positioning opening 50 and the landing surface 30 is repositioned to engage the bone landmarks at approximately 90° to the anterversion positioning of the landing surface 30. In one embodiment, the landing surface 30 is repositioned to engage the teardrop and superior edge. The position of the alignment guide 10 is then adjusted in reference to the anteversion guide 64 by tilting the handle 15 to align with the anteversion guide 64. In this way, the placement of the combined guide 66 is determined and its positioning on the subject's pelvic bone represents the angular orientation with respect to both inclination and anteversion.

According to one embodiment (FIG. 9), the alignment guide 10 may utilize a single guiding member 40 for positioning the combined guide 66. In this embodiment, the alignment guide 10 is first positioned on the respective bone landmarks in anteversion (as discussed above). Once the anteversion guide 64 is set in place, the guiding member 40 can be slidingly translated through the positioning member 20 and the landing surface 30 repositioned on the respective bone landmarks, approximately 90° to the anteversion positioning. Sliding translation of the guiding member 40 allows the combined guide 66 to be received by the corresponding positioning opening 50 at the end opposite to what was used for positioning the anteversion guide 64 in order to facilitate placement of the combined guide 66 at the targeted site.

In a further embodiment (FIG. 15), the alignment guide 10 may utilize two guiding members 40 for positioning the combined guide 66. In this embodiment, once the anteversion guide 64 is set in place, the guiding member 40 is removed from the positioning member 20 and coupled to the positioning member 20 at its opposite end, i.e., in the opposing barrel of the alignment guide 10. In this way, when the landing surface 30 is repositioned on the respective bone landmarks, approximately 90° to the anteversion positioning, the combined guide 66 can be positioned at the targeted site.

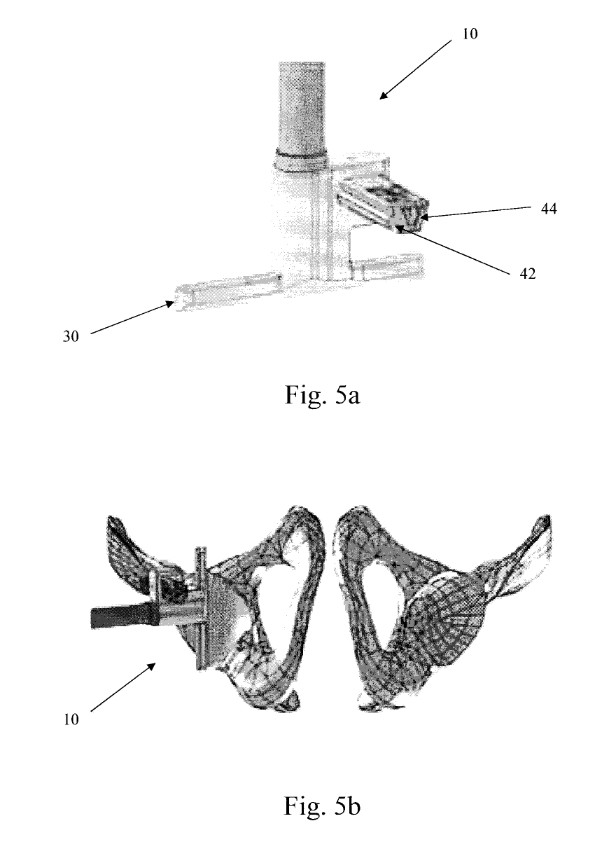

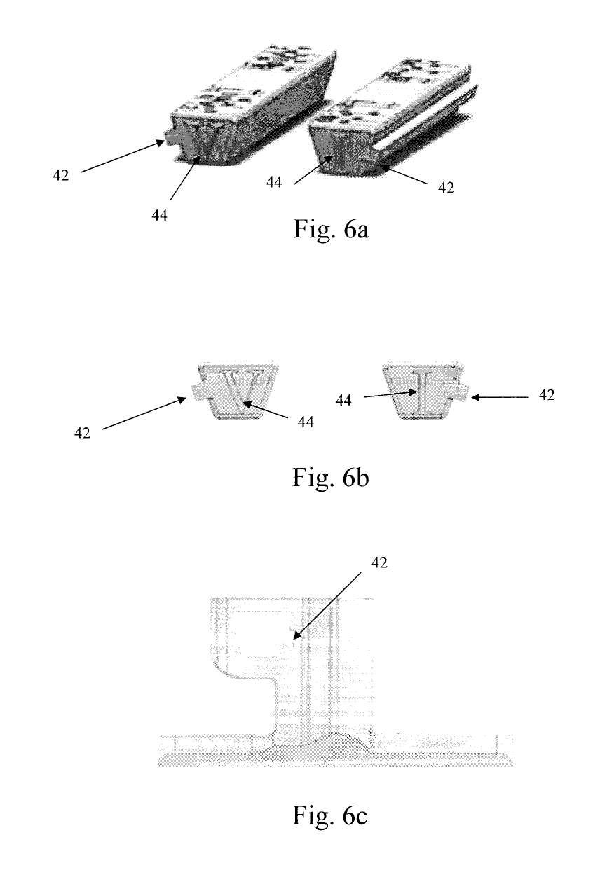

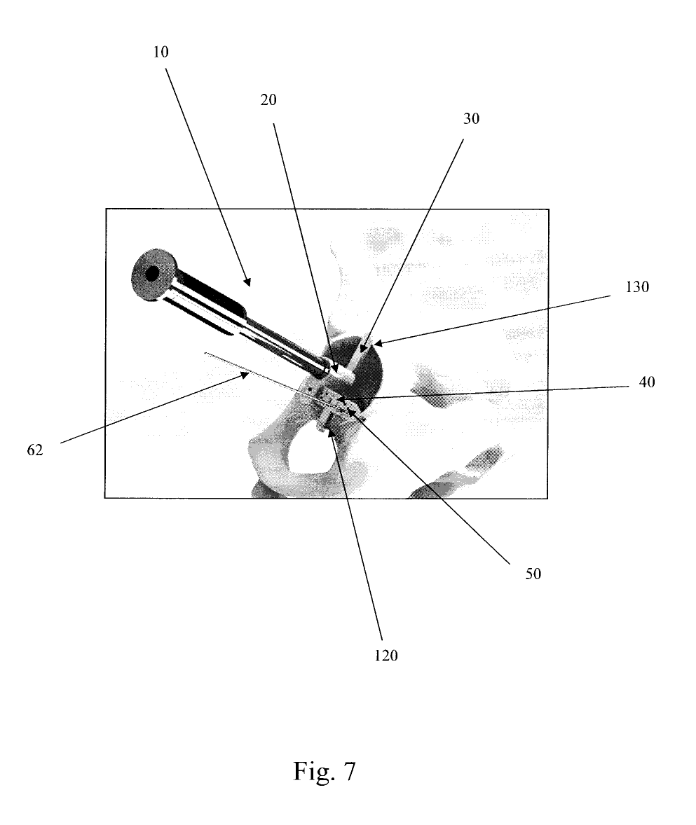

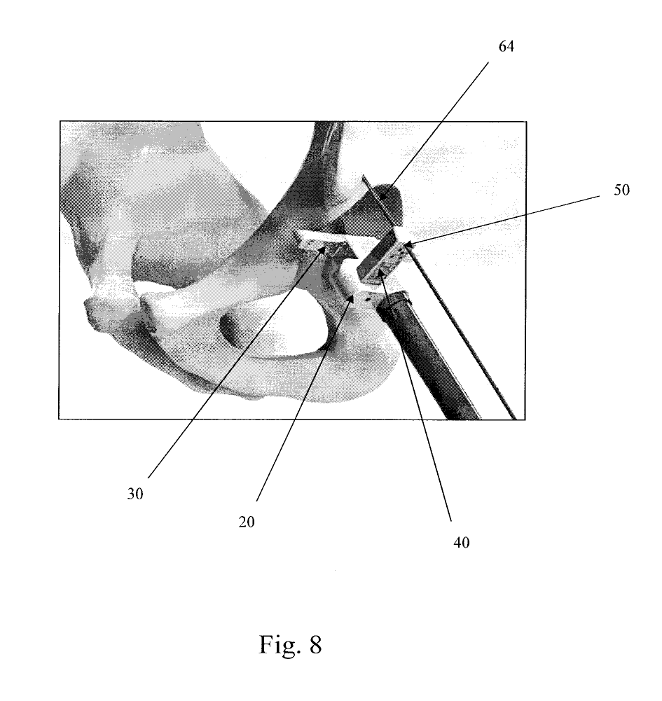

In some embodiments, the guiding members 40 comprise indicator means in order to ensure that the correct direction is used for determining anteversion and combined positioning, respectively (FIGS. 4 and 5). In this way, user confusion is avoided. For example, as shown in FIG. 6, the guiding members 40 may comprise keyways 42 that matingly engage with the positioning member 20 to ensure that the correct direction is used. In other embodiments, the guiding members 40 may further comprise indicia 44 at each respective end to indicate the correct direction for anteversion (e.g., “V”) or combined/inclination (“I”).

The combined guide 66 is then fixed into place on the subject's pelvic bone and the alignment guide 10, and optionally the anteversion guide 64, can then be removed. The remaining combined guide 66 is then left as a visual reference for angular orientation, specifically both inclination and anteversion orientation. In a preferred embodiment, the remaining combined guide 66 acts as a visual reference for positioning the acetabular component into the acetabular socket with respect to both inclination and anteversion orientation.

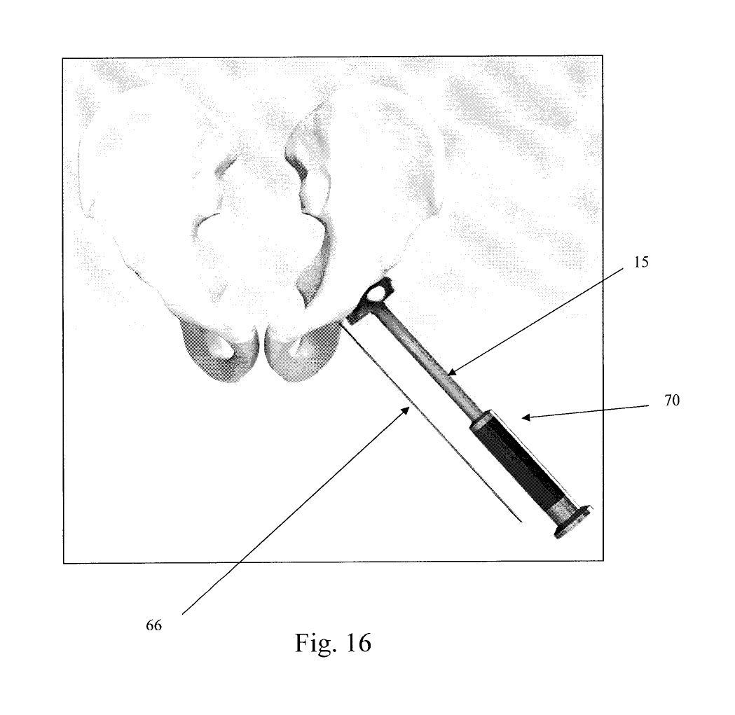

In some embodiments, as shown in FIG. 14, an alignment indicator member 90 can be used to facilitate alignment with the position guide 60 (an anteversion guide 64 in this example). The alignment indicator member 90, in some embodiments, is a flag. In further embodiments, the flag is calibrated, comprising a series of parallel indicia, for example the indicia can comprise a series of parallel-lines or cut-outs. In other embodiments, the alignment indicator member 90 is rigid for increased durability. Accurate alignment with the position guide 60 is facilitated by aligning the handle 15 of the alignment guide 10 with the indicator member 90. For example, in some embodiments, the handle 15 can be aligned with the flag itself or the calibrations, e.g., parallel lines, on the indicator member 90. In this way, alignment with the position guide 60 can be accurately achieved visually without unnecessary handling of the position guide 60.

### Inserting the Acetabular Component

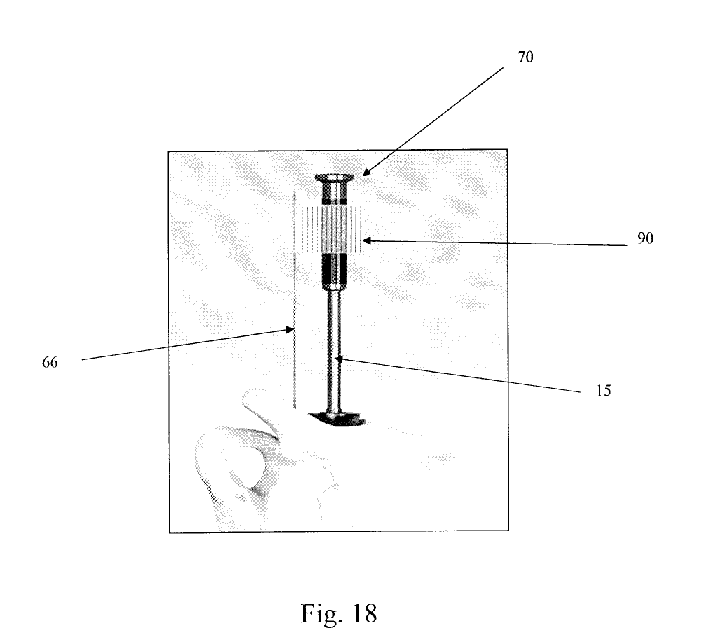

The alignment guide 10 and the method of the present disclosure provide a visual reference for positioning an acetabular component in hip arthroplasty. Specifically, once the angular orientation is fixed by the position guide 60 at the acetabular site, the acetabular component can be inserted into position by using the position guide 60 to visually guide the angle, and in some embodiments the depth, of the implant inserter 70, as shown in FIG. 16. In this way, the present disclosure further facilitates a system for determining the angular orientation, and for positioning an acetabular component, at the determined orientation. In some embodiments, the handle 15 interchangeably couples to both the positioning member 20, when positioning the angular orientation, and the implant inserter 70, when inserting the acetabular component into position in the acetabular socket.

In alternative embodiments, it is contemplated that the position guide 60 can be used to visually position other apparatuses at the acetabular socket. For example, in one embodiment, the position guide 60 can be used as a visual guide for positioning a reamer at the desired angular orientation for reaming the acetabular socket prior to insertion of the acetabular component.

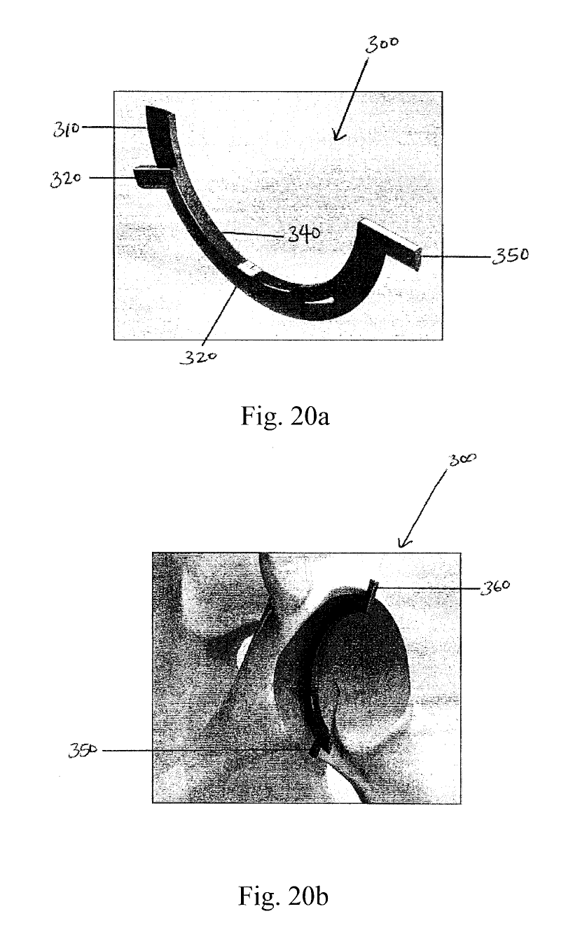

In some embodiments, as illustrated in FIG. 18, an alignment indicator member 90 can be used to facilitate the alignment of the implant inserter 70 with the position guide 60 (a combined guide 66 as shown for example). The alignment indicator member 90, in some embodiments, is a calibrated flag comprising a series of parallel indicia, for example the indicia can comprise a series of parallel-lines or cut-outs. By aligning the handle 15 of the implant inserter 70 with the parallel lines of the alignment indicator member 90, or simply with the face of the parallel alignment guide, alignment with the position guide 60 can be accurately achieved visually and without unnecessary handling of the position guide 60.

### Depth Positioning

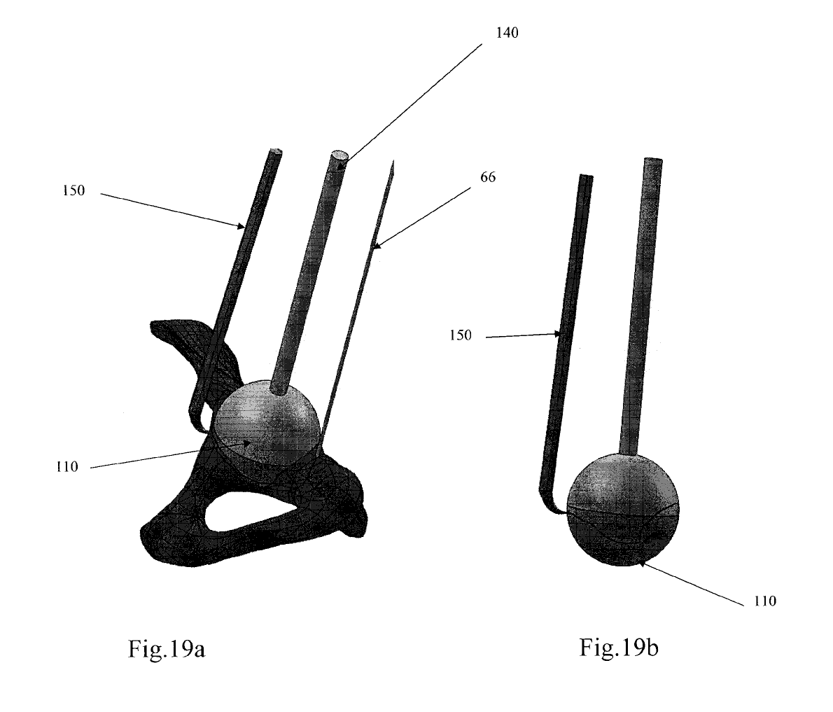

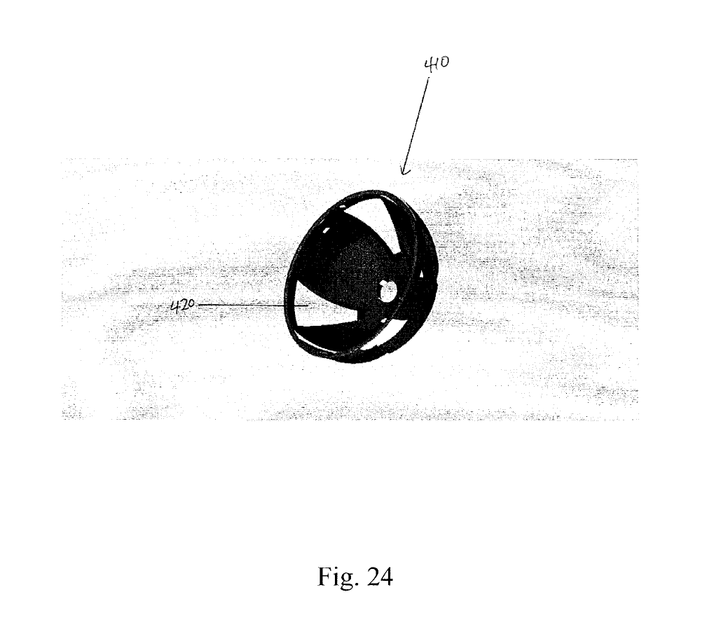

According to embodiments of the present disclosure, the depth positioning of the acetabular component can further be established during the hip arthroplasty procedure. Referring to the embodiment illustrated in FIG. 17, a trial implant inserter 72, having an acetabular cup comprising openings to allow bone visibility (FIG. 24), and a final implant inserter 74 are aligned using marker or tape or another means for marking 82 (FIGS. 17(a)-(c)). A depth gauge 80 is attached to the position guide 60 and the trial inserter 72 is inserted into the surgical site using the position guide 60 as an alignment guide. In the embodiment shown in FIG. 17 (b and c), the depth gauge 80 is a calibrated flag. Once at the desired orientation and depth, the position of the trial inserter 72 handle mark 82 is noted relative to the depth gauge 80. The trial inserter 72 is then removed and the final implant inserter 74 is inserted with the acetabular component, again using the position guide 60 to guide orientation. The final implant inserter 74 is inserted until reaching the noted position on the depth gauge 80 (FIG. 17(c)). The acetabular component is then secured into place in the acetabular socket and the final implant inserter 74, position guide 60 and depth gauge 80 removed.

In some embodiments, a single inserter may be used to determine both the trial depth and final insertion of the implant. For example, the trial 72 and final 74 inserters may be the same device, in which case a mark or feature can be noted and the same depth achieved with the final cup insertion as for the trial cup insertion.

### Verification

### Rim Verification

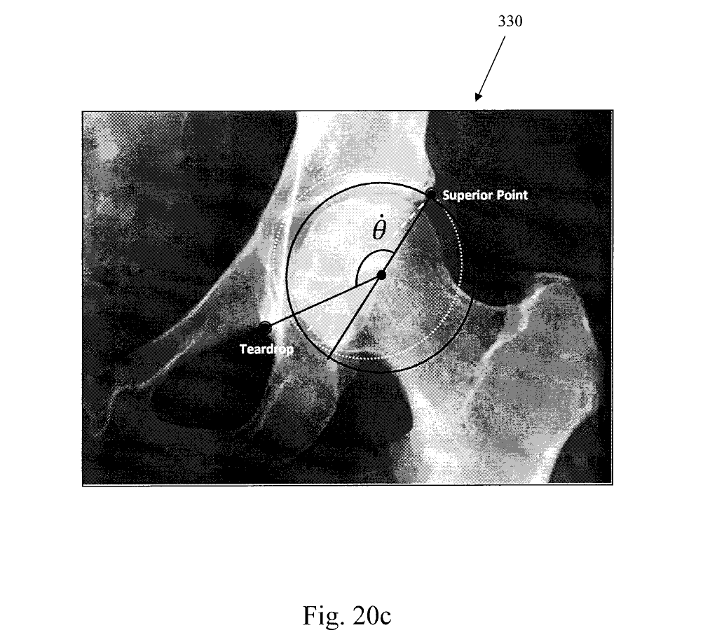

In some embodiments of the present disclosure, the positioning of the acetabular component to be implanted in the acetabular socket of the subject can be further verified. In one embodiment, the positioning of the acetabular component relative to the subject's acetabular rim can be evaluated in order to verify the positioning of the component before being press-fit into place, for example. In this way, the positioning can be verified and further adjustments may be made before the component is permanently positioned in place. FIGS. 19 (a) and (b) illustrate perspective views of a device according to one embodiment of the present disclosure, for evaluating and verifying the positioning of the acetabular component relative to the acetabular rim. The rim verification device 100 includes a spherical component 110 corresponding in size to the acetabular reamer used in the procedure. The hemispherical equator of the spherical component 110 is marked around the circumference of the spherical component 110 as a reference indicator. The spherical component 110 further has an elongate handle 140 extending therefrom to allow alignment with a position guide 60 and insertion of the spherical component 110 into the acetabular socket.

In the embodiment illustrated in FIG. 19(a), the device handle 140 is aligned with the combined guide 66 fixed in the pelvic bone of the subject. The spherical component 110 is inserted into position in the acetabular socket in reference to the combined guide 66. Once in position, a marking tool 150 is used to trace the acetabular rim of the socket onto the surface of the spherical component 110 which can then be evaluated to verify that the positioning of the implant corresponds with the preoperative and surgical plan. Adjustments to the positioning can then be undertaken if necessary.

The marking tool used to trace the acetabular rim of the socket can be adapted to facilitate access to the target site. In one embodiment, the marking tool has an angled tip to facilitate access to the target site. In a further embodiment, the marking tool is an electrocautery tool. In another embodiment, the marking tool is a marker pen.

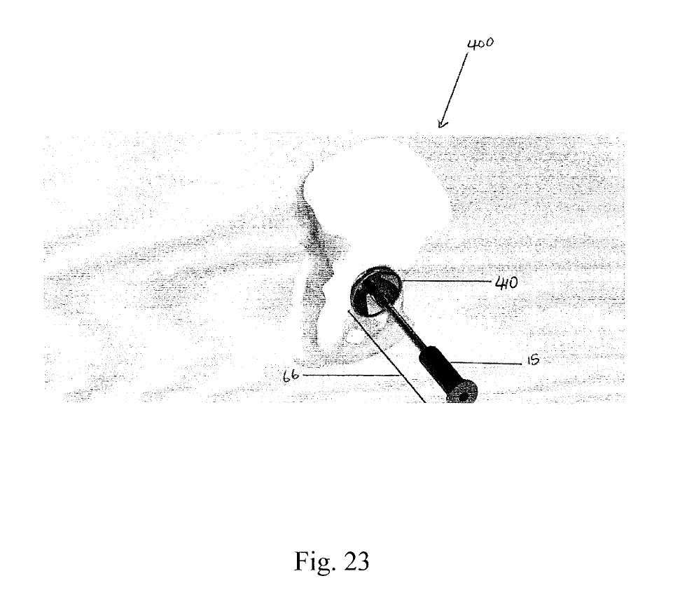

In a further embodiment, as shown in FIGS. 23 and 24, visual verification of the positioning of the acetabular component in the acetabular socket can be achieved without any marking tool. As shown in FIG. 23, the verification device 400 according to this embodiment comprises a cup 410 coupled to a handle 15 at one end. In some embodiments, the trial implant inserter 72 itself, as described above, can also be used to visually verify the positioning of the acetabular component. In this way, the verification device may in certain embodiments be considered optional. The cup 410 has substantially similar dimensions to the final acetabular component, but having a diameter that matches the reamed diameter as opposed to the press-fit diameter. The cup 410 further comprises visibility openings 420 to allow visibility of the acetabular cavity and assurance of full seating in the acetabular socket. In operation, the verification device 400 is positioned such that its handle 15 is aligned parallel to the position guide 60 and in this way the position and orientation of the acetabular component can be visualized to provide a secondary check of the suitability of the cup 410 placement before inserting the final acetabular component.

### Reamed Depth Verification

In further embodiments of the present disclosure, the reamed depth of the acetabular socket can also be evaluated prior to permanently positioning the acetabular component. In this way, the reamed depth of the acetabular socket can be verified and any necessary adjustments can be made.

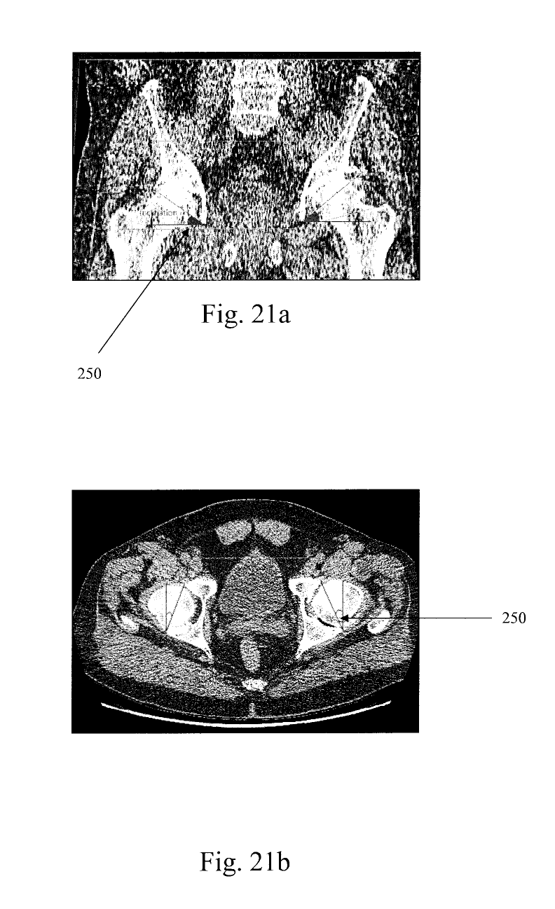

FIGS. 20 (a), and (b) illustrate perspective views of a device for evaluating the depth position of an acetabular component, according to embodiments of the present disclosure. As shown, the depth evaluation device 300 comprises two interengaging parts 310, 320 that together function as a protractor within the acetabulum. The two parts 310, 320 slidably interengage with each other to allow the device 300 to be set at a determined angle.

In operation, as shown in FIG. 20(c), the desired location of an acetabular component in an acetabulum is first templated on a preoperative X-ray 330. The expected angle (θ) on the hemispherical implant template is then measured on the X-ray, up to the teardrop. As shown in FIG. 20(b), the depth evaluation device 300 is then set by sliding the interengaging parts 310, 320 until the calculated angle (θ) is reached on the corresponding scale 340. The set depth evaluation device 300 is then positioned in the reamed acetabulum during surgery. If the angle (θ′) is smaller than the expected angle (θ) measured on the template, then the reamed acetabulum is not deep enough and adjustment can then be made.

Alternatively, the evaluation device 300 can first be placed in the acetabular socket such that the two parts 310, 320 of the protractor can slide freely within the acetabular socket until the extensions 350 and 360 at each respective end engages with the teardrop and superior rim. The resulting angle (θ′) is then measured on the scale 340 and compared to the expected preoperative angle (θ) to determine whether further depth adjustment is needed.

It is contemplated that any embodiment discussed herein can be implemented with respect to any method or composition of the invention, and vice versa. Furthermore, compositions and kits of the invention can be used to achieve methods of the invention.

The disclosures of all patents, patent applications, publications and database entries referenced in this specification are hereby specifically incorporated by reference in their entirety to the same extent as if each such individual patent, patent application, publication and database entry were specifically and individually indicated to be incorporated by reference.

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

1. An apparatus for positioning an acetabular component during a hip arthroplasty procedure, the apparatus comprising:
a positioning member for engaging an acetabular socket, said positioning member having a landing surface for engaging said acetabular socket relative to at least one bone landmark; and
an elongate guiding member coupleable to said positioning member about perpendicular to said landing surface, said guiding member adjustable to a plurality of positioning angle settings and configured to receive a position guide;
wherein adjustment of said guiding member to said positioning angle setting orients said position guide onto a target site at said acetabular socket.

2-4. (canceled)

5. The apparatus according to claim 1, wherein said elongate guiding member is rotatable relative to said positioning member, wherein rotation of said guiding member corresponds to said plurality of positioning angle settings.

6. The apparatus according to claim 1, wherein said elongate guiding member comprises a plurality of openings along the length of said guiding member for receiving said position guide, said plurality of openings corresponding to said plurality of positioning angle settings.

7. The apparatus according to claim 6, wherein said plurality of positioning angle settings is fixed at increasing and/or decreasing increments ranging from about 1° to about 5°.

8-9. (canceled)

10. The apparatus according to claim 6, wherein said guiding member further comprises two opposing ends, each end comprising said plurality of openings; said first end comprising positioning openings corresponding to positioning angle settings for a right hip acetabular socket and said second end comprising positioning openings corresponding to positioning angle settings for a left hip acetabular socket of a subject, whereby the positioning angle setting for the right or left hip acetabular socket is selected by slidable translation of said guiding member relative to said positioning member.

11. The apparatus according to claim 1, wherein said guiding member slidably translates about perpendicularly to said positioning member relative to a geometry of said acetabular socket, whereby the slidable translation allows engagement of a plurality of acetabular sockets wherein each of the plurality of acetabular sockets has a different shape and size relative to the others of the plurality of the acetabular sockets.

12. The apparatus according to claim 1, wherein said landing surface of the positioning member is V-shaped.

13. The apparatus according to claim 1, wherein said landing surface ranges in length from about 40 to about 90 mm.

14-15. (canceled)

16. The apparatus according to claim 1, wherein said position guide comprises an alignment indicator member couplable to said position guide, said alignment indicator member having a calibrated scale for guiding alignment of an implant inserter parallel to said position guide when inserting the acetabular component into position in the acetabular socket of a subject.

17. (canceled)

18. The apparatus according to claim 1, additionally comprising a crosshair for marking the at least one bone landmark on the acetabular rim of said socket to guide the engagement of said landing surface on said socket, wherein the crosshair comprises two, three, or four arms for correspondingly marking the at least one bone landmark on the acetabular rim of said socket.

19-21. (canceled)

22. The apparatus according to claim 1, wherein said position guide comprises a depth gauge having a calibrated scale for aligning an implant inserter to a desired depth of insertion in the acetabular socket of said subject.

23-24. (canceled)

25. A method for positioning an acetabular component for implantation during a hip arthroplasty procedure performed on a subject, the method comprising:
a) determining a positioning angle from a radiographic image, said positioning angle determined relative to predefined landmarks at the acetabular socket of the subject's pelvis; and
b) engaging the apparatus of claim 1 with the acetabular socket of said pelvis relative to said predefined landmarks, said apparatus having a first position guide coupled thereto, whereby the orientation of said first position guide corresponds to said positioning angle;
c) inserting said first position guide to the pelvis of said subject.

26. The method according to claim 25, wherein said first position guide defines an inclination orientation for said acetabular socket.

27. The method according to claim 25, wherein said first position guide defines an anteversion orientation for said acetabular socket.

28. The method according to claim 25, wherein said first position guide defines both an anteversion and an inclination orientation for said acetabular socket.

29. The method according to claim 25, further comprising
d) coupling a second position guide to said apparatus and engaging said apparatus with the acetabular socket of said pelvis relative to said first position guide, whereby said second position guide is inserted into the pelvis of said subject; and
e) optionally removing said first position guide such that said second position guide remains in position at the acetabular socket for guiding said positioning.

30-31. (canceled)

32. The method according to claim 29, wherein said first position guide defines an inclination orientation for said acetabular socket and said second position guide defines anteversion orientation for said acetabular component.

33. (canceled)

34. The method according to claim 29, wherein said first position guide is removed and said second position guide defines both inclination and anteversion orientation for said acetabular component.

35-36. (canceled)

37. The method according to claim 29, further comprising:
f) aligning an implant inserter to said second position guide, said implant inserter comprising said acetabular component;
wherein said acetabular component is positioned in reference to said second position guide for implantation in said acetabular socket.

38. The method according to claim 37, further comprising:
g) positioning said implant inserter at a desired depth of insertion in the acetabular socket of said subject, wherein said depth positioning is in reference to a depth gauge attached to said second position guide.

39-40. (canceled)

41. A device for evaluating the reamed depth of an acetabular socket, the device comprising two slidably interengaging parts, each part comprising at one end an extension for engaging with a respective landmark at said acetabular socket, wherein said interengaging parts together function as a protractor for determining an angle when said extensions are engaged with said landmarks and said device is positioned in said acetabular socket.

42. (canceled)

43. A device for guiding depth positioning of an acetabular component, the device comprising a depth gauge having a calibrated scale for aligning an implant inserter to a desired depth of insertion in the acetabular socket of a subject, wherein the depth gauge is attachable to a position guide.

44-45. (canceled)
